(19) United States
(12) Patent Application Publication
(10) Pub. No.: US 2023/0235375 A1
(43) Pub. Date: Jul. 27, 2023

(54) FERMENTATION PROCESS

(71) Applicant: DSM IP ASSETS B.V., Heerlen (NL)

(72) Inventors: Eric PIRON, Kaiseraugst (CH); Michael HANS, Kaiseraugst (CH); Maarten Job VAN DE GRAAF, Kaiseraugst (CH)

(21) Appl. No.: 18/002,577

(22) PCT Filed: Jun. 23, 2021
(86) PCT No.: PCT/EP2021/067231

§ 371 Date: Dec. 20, 2022

(30) Foreign Application Priority Data
Jun. 23, 2020 (EP) 20181523.0

(51) Int. Cl. C12P 25/00; C12N 1/20; C12P 7/66

(52) U.S. Cl. CPC C12P 25/00; C12N 1/20; C12P 7/66; C12R 2001/125

(57) ABSTRACT

The present invention is related to sustainable fermentation processes with increased efficiency and less environmental impact. Particularly, the present invention is related to a process wherein in one fermentation process two or more fermentation products can be produced and isolated, i.e. a “primary” fermentation product and a “secondary” fermentation product, particularly wherein one is a water soluble organic compound and one is a fat-soluble organic compound particularly a fat-soluble vitamin, preferably vitamin K2.

FERMENTATION PROCESS

The present invention is related to sustainable fermentation processes with increased efficiency and less environmental impact. Particularly, the present invention is related to a process wherein in one fermentation process two or more fermentation products can be produced and isolated, i.e. a “primary” fermentation product and a “secondary” fermentation product, particularly wherein one is a water soluble organic compound and one is a fat-soluble organic compound particularly a fat-soluble vitamin, preferably vitamin K2.

Many products including vitamins, enzymes, antibiotics, amino acids, fuels are nowadays generated by large-scale fermentation rather than produced via chemical synthesis. A few of multiple advantages of biotechnology is the use of less-toxic ingredients leading to more healthy and natural or bio-based products. Typically, in such fermentative processes a host cell (including genetically modified host cells expressing heterologous genes to enable production of the fermentation product) is cultivated under suitable conditions, and the fermentation product is extracted/isolated from the biomass. The biomass is mostly being “disposed” or discarded, eventually after specific treatment of said biomass to eliminate recombinant DNA still present and being harmful to the environment.

Although biotechnological processes could lead to more bio-based products, extraction of fermentation products from the cultivation medium might still have some negative impact either with regards to economic and ecological aspects: for instance, isolation of many fermentation products, such as e.g. vitamin K2, requires drying of the biomass before efficient extraction, such as e.g. via supercritical CO₂ as known in the art, thus a procedure that is typically very expensive. Use of solvents including hexane or heptane, that are commonly used in the field, can also have some negative impact on environment and health (being a no-go for food applications) and thus should be avoided.

On top of this, and especially to reach the UN Sustainable Development Goals there is a strong need for reduction of waste and greenhouse gas emission, i.e. to look for more eco-friendly and sustainable production processes, including recycling of biomass and/or extraction of valuable compounds from said biomass in a sustainable way using non- or less toxic procedures as compared to the known processes.

Surprisingly, we now found a way of optimizing fermentative production of organic compounds, wherein with one fermentation process using one (common) host cell at least two valuable fermentation products could be obtained, i.e. a “primary” fermentation product and a “secondary” fermentation product, particularly wherein one of the fermentation products is water-soluble and one fermentation product is fat-soluble.

Particularly, the present invention is directed to a co-fermentation process in a suitable host cell, said host cell being capable of co-production of at least two fermentation products, with one fermentation product being water-soluble and one fermentation product being fat-soluble, particularly a fat-soluble vitamin, preferably vitamin K, more preferably vitamin K2. Preferably, the primary fermentation product is water-soluble and the secondary fermentation product is fat-soluble.

As used herein, the term “one fermentation process” means cultivation of a suitable host cell under suitable culture conditions in a way that the primary fermentation product is formed, said primary fermentation product is isolated from the production stream, i.e. separated from the biomass, and additionally, the second fermentation product is extracted from said biomass. The terms “one fermentation process” and “co-fermentation process” is used interchangeably herein. This might also include co-production of at least two fermentation products using one host cell, wherein both fermentation products are simultaneously extracted or isolated and separated afterwards.

As used herein, a “valuable fermentation product” might be selected from both water- and/or fat-soluble organic compounds selected from vitamins, enzymes, oligosaccharides or amino acids, particularly wherein one such fermentation product is a water-soluble compound and one such fermentation product is a fat-soluble compound. Suitable enzymes within the scope of the present invention might be selected from proteases, amylases, glucosidases, cellulase or the like. Suitable amino acids within the scope of the present invention might be selected from lysine, tryptophan or monosodium glutamate. Suitable vitamins within the scope of the present invention might be selected from water-soluble or fat-soluble vitamins, such as e.g. vitamin B2 (riboflavin), vitamin B5, vitamin B6, vitamin B12, vitamin B3, vitamin B1, vitamin D, vitamin K or vitamin B7, particularly vitamin B2, B5, B6, B12, vitamin K, particularly vitamin K2, more particularly vitamin B2 and vitamin K2.

In one embodiment, the present invention is directed to a co-fermentation process wherein at least one or two vitamins are co-produced, particularly wherein at least one of the fermentation products is a water-soluble vitamin and one of the fermentation products is a fat-soluble vitamin, particularly vitamin K, preferably vitamin K2. Particularly, the primary fermentation product is selected from water-soluble vitamins, such as vitamins selected from the group consisting of vitamin B2, vitamin B5, vitamin B6, vitamin B12, vitamin B3, vitamin B1 or vitamin B7, particularly vitamin B2, B5, B6, B12, more particularly vitamin B2. In a preferred embodiment, the primary fermentation product is vitamin B2 produced in Bacillus, such as e.g. B. subtilis (for biosynthesis of vitamin B2 see e.g. EP405370, FIG. 2 in EP1186664 or Ullman's Encyclopedia of Industrial Chemistry, 7^{th} Edition, 2007, Chapter Vitamins).

Thus, in a preferred embodiment the primary fermentation product is a water-soluble vitamin, more preferably a vitamin selected from vitamin B1, B2, B3, B5, B6, B7, B12, most preferably vitamin B2.

In a further embodiment, the present invention is directed to a co-fermentation process wherein at least one or two vitamins are co-produced, particularly wherein at least one of the fermentation products is a fat-soluble vitamin, particularly wherein the fat-soluble vitamin is produced as secondary fermentation product, wherein said secondary fermentation product is extracted from the biomass or “production stream” of the primary fermentation product, i.e. wherein the secondary fermentation product is extracted from the biomass that is generated during the fermentative production of the primary fermentation product, particularly the primary fermentation product being selected from water-soluble vitamins as defined above. An example of such fat-soluble vitamin is vitamin K, wherein vitamin K2, also known as menaquinone, as one of the natural forms is preferred, more preferably vitamin K2 comprising MK-7, MK-4, MK-6, MK-3 as isoforms with the percentage of MK-7 being at least about 80% (wt/wt) as compared to the other isoforms.

As used herein, the term “production stream” in the context of a fermentative process means mass stream comprising the desired fermentation product(s), i.e. at least the primary and secondary fermentation products as defined herein.

Thus, the present invention is directed to co-production of at least two fermentation products, wherein one of the fermentation products is vitamin K2 produced as secondary fermentation product, such as e.g. via extraction or isolation from the production stream of the primary fermentation process as defined herein, said vitamin K2 comprising a percentage of MK-7 of at least about 80% (wt/wt), such as e.g. in the range of at least about 85, 90, 92, 95, 96, 98, 99 or even 100% (wt/wt) MK-7.

In a further aspect, the present invention is directed to co-production of at least two fermentation products, wherein one of the fermentation products is vitamin K2 produced as secondary fermentation product, such as e.g. via extraction or isolation from the production stream of the primary fermentation process as defined herein, said vitamin K2 comprising a percentage of DNA, particularly recombinant DNA, of 10 ng DNA or less per g of fermentation product, such as vitamin K2, preferably wherein said vitamin K2 comprising a percentage of MK-7 of at least about 80% (wt/wt), such as e.g. in the range of at least about 85, 90, 92, 95, 96, 98, 99 or even 100% (wt/wt) MK-7.

Thus, the present invention is particularly useful in a fermentation process using genetically modified or recombinant microorganisms, with removal of recombinant DNA to a final concentration in the range of about 10 ng/g fermentation product, such as e.g. bio-based vitamin K2 as the secondary fermentation product as defined herein, as measured by known PCR methods.

In one embodiment, the present invention is directed to co-production of at least two fermentation products, wherein one of the fermentation products is vitamin K2 produced as secondary fermentation product, such as e.g. via extraction or isolation from the production stream of the primary fermentation process as defined herein, said vitamin K2 comprising a percentage of production strain, particularly recombinant production strain, of 1 colony forming unit (CFU) per 1 g of fermentation product, such as vitamin K2, preferably wherein said vitamin K2 comprising a percentage of DNA, particularly recombinant DNA, of 10 ng DNA or less per g of fermentation product and/or comprising a percentage of MK-7 of at least about 80% (wt/wt), such as e.g. in the range of at least about 85, 90, 92, 95, 96, 98, 99 or even 100% (wt/wt) MK-7.

The CFU is typically measured as known in the art, e.g. visually counted by plating out a product on an agar-plate, incubating the agar plate under representative conditions allowing growth of the production strain and subsequently counting the amount of colonies on the plate, in combination with confirming that the colonies are the production strain (by DNA sequencing).

A suitable host cell might be selected from a strain of Lactococcus, Lactobacillus, Enterococcus, Leuconostoc, Streptococcus, Bacillus, Corynebacterium, Pseudomonas, Flavobacterium, Elizabethingia or Escherichia, preferably selected from Bacillus, more preferably from B. amyloliquefaciens, B. subtilis, B. licheniformis, B. subtilis natto, B. polymyxa, B. firmus, B. megaterium, B. cereus, B. thuringiensis, or from a strain selected from Flavobacterium so., Flavobacterium meningosepticum, Elizabethingia meningoseptica, E. coli, Lactococcus lactis ssp. lactis, Lactococcus lactis ssp. cremonis, Flavobacterium meningosepticum, Flavobacterium sp. or C. glutamicum. Most preferably, the host cell is selected from Bacillus, particularly B. subtilis.

Thus, the present invention is directed to co-fermentation as described above, wherein at least a primary and a secondary fermentation product is produced, particularly a water-soluble vitamin as e.g. vitamin B2 together with a fat-soluble vitamin as e.g. vitamin K2 as defined herein, said co-fermentation being performed in a suitable host cell selected from the group consisting of Lactococcus, Lactobacillus, Enterococcus, Leuconostoc, Streptococcus, Bacillus, Corynebacterium, Pseudomonas, Flavobacterium, Elizabethingia and Escherichia, preferably selected from B. amyloliquefaciens, B. subtilis, B. licheniformis, B. subtilis natto, B. polymyxa, B. firmus, B. megaterium, B. cereus, B. thuringiensis, Flavobacterium sp., such as Flavobacterium meningosepticum, Elizabethingia meningoseptica, E. coli, Lactococcus lactis ssp. lactis, Lactococcus lactis ssp. cremonis, Flavobacterium meningosepticum, or Flavobacterium sp. or C. glutamicum. Most preferably, the host cell is Bacillus subtilis.

In a preferred embodiment the invention includes fermentative production of vitamin B2 as the primary fermentation product in B. subtilis, such as e.g. B. subtilis RB50::[pRF69]ₙ containing multiple (n) copies (for example about 5 to about 20 copies) of pRF69 encoding a rib operon modified with the strong promoter Pₛₚₒ₁₅ to enhance transcription of the rib genes (see e.g. EP405370 or Perkins et al., J. Ind. Microbiol. Biotechnol., 22:8-18, 1999 for construction of the strain and culture conditions to result in vitamin B2 production). In order to increase production of vitamin B2, the strain might carry further modifications such as e.g. overexpression of one or more riboflavin biosynthetic gene(s), in particular ribA, introduction of multiple copies of the rib operon in the host cell, such as implemented in B. subtilis strain RB50 (see e.g. EP405370), fusion of the rib operon to a strong promoter, heterologous expression of pyridoxal phosphatase [EC 3.1.3.74], decoupling of growth from production of riboflavin, such as e.g. via introduction of an auxotrophy such as described in EP1186664 for e.g. biotin, and/or furthermore combined with introduction of modified transketolase gene as e.g. described in WO2007051552, and/or furthermore combined with the use of modified rib leader sequences as e.g. described in WO2010052319. The skilled person knows how to manipulate a production strain in order to increase production of the respective fermentation product, particularly vitamin B2 being the primary fermentation product, and to use this in a co-fermentation as described herein, particularly with vitamin K2 as the secondary fermentation product.

The term “waste biomass” or “non-valuable waste stream” as used herein defines the remaining biomass after recovery of at least the primary and secondary fermentation product, that might be disposed or further used as fertilizer or for extraction of further valuable organic compounds.

In one particular embodiment, the present invention is directed to co-fermentation of a primary and a secondary fermentation product as defined herein, wherein either both or only one fermentation product(s) is/are produced inside the cell or in the cell membrane or wherein at least one of the fermentation products, particularly the primary fermentation product, is secreted outside the host cell in the form of crystals. Particularly, the primary fermentation product is secreted outside the cell, such as e.g. vitamin B2, whereas the secondary fermentation product is produced in the cell membrane and thus to be recovered from the biomass (i.e. from the production stream of the fermentation process as defined herein) as secondary fermentation product, such as e.g. vitamin K2.

In one particular embodiment, the process according to the present invention comprises several steps, including but not limited to:

(a) cultivation of the host cell under suitable conditions and in a suitable culture medium depending on the primary fermentation product, particularly wherein the primary fermentation product is selected from vitamins, amino acids, enzymes or oligosaccharides, preferably from water-soluble vitamins, more preferably from vitamin B1, B2, B3, B5, B6, B7 or B12, most preferably from vitamin B2,
(b) isolation of said primary fermentation product from the production stream, i.e. separation of the primary fermentation product from the biomass,
(c) recovery of the secondary fermentation product, particularly fat-soluble vitamin such as vitamin K, particularly vitamin K2 as defined herein, from the production stream of the fermentation process, i.e. from the biomass generated during fermentation/production of the first fermentation product, and optionally
(d) purification of the fermentation product(s).

Optionally, the waste biomass from step (d) might be disposed or used for extraction of further valuable organic compounds.

In one embodiment, the process according to the present invention provides fermentation products (i.e. primary and secondary fermentation products) both in solid form, such as e.g. crystalline form. In one example, the present invention is directed to co-production of vitamin B2 (as the primary solid fermentation product) and vitamin K2 (as the secondary solid fermentation product), i.e. wherein both products are solid, with one fermentation product being excreted outside the host cell and one being accumulated inside the cell.

In a further embodiment, the process according to the present invention provides fermentation products (i.e. primary and secondary fermentation products), with one of the product being in solid form and the other product being in liquid form and wherein the liquid fraction might be separated from the biomass/production stream.

The co-fermentation process as defined above might be performed in batch, fed-batch or continuous mode, e.g. depending on the primary fermentation product, including but not limited to production of vitamin B2, more preferably in a process using Bacillus subtilis as host cell according to EP405370 or WO2007051552.

Cultivation of a suitable host cell as defined above may vary depending on for instance the host, the primary fermentation product, pH, temperature and nutrient medium to be used. In the case of riboflavin production with B. subtilis as host cell, the process can be performed for about 10 h to about 10 days, preferably for about 4 to about 7 days, more preferably for about 2 to about 6 days, depending on the microorganism. The skilled person knows the optimal culture conditions of suitable microorganisms/host cells to be used in connection with the present invention.

In a particular embodiment, the co-fermentation process according to the present invention may be conducted for instance at a pH of about 7.0, preferably in the range of about 6 to about 8, more preferably about 6.5 to 7.5. A suitable temperature range for carrying out the cultivation may be for instance from about 13° C. to about 70° C., preferably from about 30° C. to about 39° C., more preferably from about 35° C. to about 39° C., and most preferably from about 36° C. to about 39° C. The culture medium for growth usually may contain such nutrients as assimilable carbon sources, including compounds consisting of 3, 5 or 6 carbon atoms, such as e.g. D-glucose, glycerol, thick juice, dextrose, starch, sucrose or ribose; and digestible nitrogen sources such as organic substances, e.g., peptone, yeast extract and amino acids. The media may be with or without urea and/or corn steep liquor and/or baker's yeast. Various inorganic substances may also be used as nitrogen sources, e.g., nitrates and ammonium salts. Furthermore, the growth medium usually may contain inorganic salts, e.g., magnesium sulfate, manganese sulfate, potassium phosphate, and calcium carbonate. Cells obtained using the procedures described above can then be further incubated at essentially the same modes, temperature and pH conditions as described above, in the presence of substrates such as described above in such a way that they convert these substrates into the desired (primary) fermentation products including vitamins, oligosaccharides, amino acids or enzymes, such as e.g. water soluble vitamins, preferably vitamin B2. Incubation can be conducted in a nitrogen-rich medium, containing, for example, organic nitrogen sources, e.g., peptone, yeast extract, baker's yeast, urea, amino acids, and corn steep liquor, or inorganic nitrogen sources, e.g., nitrates and ammonium salts, in which case cells will be able to further grow while producing the desired primary fermentation products. Alternatively, incubation can be conducted in a nitrogen-poor medium, in which case cells will not grow substantially, and will be in a resting cell mode, or biotransformation mode. In all cases, the incubation medium may also contain inorganic salts, e.g., magnesium sulfate, manganese sulfate, potassium phosphate, and calcium chloride. The skilled person will know which conditions to apply depending on the host cell and the produced fermentation product(s). An example of a suitable medium for fermentative production of riboflavin is described in WO2004113510 (VF-medium), which is particularly useful with regards to Bacillus and which might be used for the purpose of the present invention.

Depending on the fermentation products, separation of the primary fermentation product from the biomass (i.e. extraction of the product from the production stream) as defined above might be performed in different ways: in case the primary fermentation product is excreted outside the cell, such as e.g. in the form of crystals including but not limited to vitamin B2, separation might include a pasteurization step, such as e.g. via heating for 4 hours at 70° C., followed by one or more solid-liquid separation step(s) including but not limited to decantation, including recovery by centrifugation (see e.g. WO2005014594). Processing of the pasteurized broth might be done via decantation, such as e.g. as preferred in the case of vitamin B2 as primary fermentation product. The crystals, such as riboflavin crystals, might optionally be further purified according to known methods. After optional further purification steps, the material might be used as component in the food/feed/pharma or cosmetic industry. In one very specific embodiment, an amount of at least about 280 mg riboflavin per l fermentation broth can be obtained.

Recovery of the secondary fermentation product might be performed via extraction/recovery from the biomass or production stream obtained from the fermentation of the primary fermentation product, said extraction/recovery being performed in a sustainable manner, particularly wherein the extraction process is free of environmental problematic solvents such as e.g. hexane and the like.

Thus, the present invention is directed to a co-fermentation process, wherein the secondary fermentation product is isolated from the production stream, such as extracted from the biomass obtained during production of the primary fermentation product, using a hexane-free solvent, preferably solvents selected from aqueous solutions (aqueous ethanol solutions), with particularly a percentage of at least about 80% (wt/wt) ethanol, such as e.g. at least about 85, 90, 92, 94, 95, 96, 97, 98 or even 100% (wt/wt) ethanol. Further suitable solvents to be used for said step might be selected from aqueous solutions comprising isopropanol or acetone.

In one particular embodiment, the recovered primary/secondary fermentation product might be further purified by performing e.g. one or several filtration steps, including e.g. microfiltration, ultrafiltration and/or nanofiltration. In a preferred embodiment, filtration is performed using membranes with a low cut off, such as e.g. in the range of below 10 kDA. Optionally, the solvent is further evaporated and after a cooling step crystals of the /primary second fermentation product are isolated. In one very preferred embodiment, crystals of bio-based vitamin K2 as secondary fermentation product as defined herein are isolated with a percentage of MK-7 of at least about 80% (wt/wt).

The primary, such as e.g. vitamin B2, and the secondary fermentation product, such as e.g. vitamin K2, i.e. both bio-based products, might be furthermore used as nutritional product, such as e.g. a composition used as food, feed, cosmetic product, comprising such vitamin B2 and/or vitamin K2 crystals generated according to the present invention.

The present invention is related to a fermentation product, particular vitamin K2, wherein the amount of recombinant DNA is reduced, such as to a range of about 10 ng DNA or less per g fermentation product as can be measured via PCR method known in the art.

The present invention is furthermore related to a fermentation product, particular vitamin K2, wherein the amount of DNA originating from the production strain is reduced, such as to a range of below about 1 CFU per g product as can be measured via dilution, incubation and plating of the product and as known in the art.

In another aspect, the present invention relates to a fermentation product, particularly vitamin K2, comprising MK-7 with a percentage in the range of at least about 80% (wt/wt), preferably in the range of at least about 85, 90, 92, 95, 96, 98, 99 or even 100% (wt/wt) MK-7, particularly with a maximal content of DNA, preferably recombinant DNA, such as in the range of about 10 ng DNA per g vitamin K2 as measured by PCR. The secondary fermentation product such as vitamin K2 might comprise further isoforms present in minor traces, such as e.g. MK-4, MK-5, MK-6, in an amount of less than about 20, 15, 10, 5, 4, 3, 2 or 1% (wt/wt). Analysis of vitamin K2 and its isoforms can be done via HPLC as known in the art or further described in the examples.

In one aspect, the present invention is related to production of nutritional products comprising bio-based vitamin K2, such as e.g. crystals with at least about 80% (wt/wt) of MK-7 and with about 20, 15, 10, 5, 4, 3, 2 or 1% (wt/wt) or less of MK-4, MK-5, and MK-6 as defined herein, particularly preparation of compositions used as/in food, feed or cosmetic product, including solid or liquid formulations, e.g. solid particles, comprising bio-based vitamin K2 as produced according to the present invention.

The bio-based vitamin K2 as produced according to the present invention to be used in food, feed or cosmetic formulations can be mixed with one or more ingredient(s) selected from the group consisting of encapsulation agents, organic solvent(s), oils, supercritical fluids, antioxidant(s), sugar, co-crystallizing agent(s), coacervate(s), and combinations thereof, depending on the final application in the food, feed, or cosmetic industry and as known in the art.

Suitable encapsulation agents to be used in formulations comprising the bio-based vitamin K2 as defined herein include but are not limited to modified (food) starches, ascorbyl palmitate, pectin, alginate, carrageenan, furcellaran, dextrin derivatives, celluloses and cellulose derivatives (e.g. cellulose acetate, methyl cellulose, hydroxypropyl methyl cellulose), lignosulfonate, polysaccharide gums (such as gum acacia/gum arabic, modified gum acacia, TIC gum, flaxseed gum, gum Bhatti, gum tamarind and arabinogalactan), gelatine (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, and sugar ester (as well as derivatives thereof), particularly maltodextrin, modified food starch or gums, such as e.g. gum acacia.

Suitable organic solvents to be used in formulations comprising the vitamin K2 as defined herein include but are not limited to halogenated aliphatic hydrocarbons, aliphatic ethers, aliphatic and cyclic carbonates, aliphatic esters and cyclic esters (lactones), aliphatic and cyclic ketones, aliphatic alcohols or mixtures thereof. Useful examples of halogenated aliphatic hydrocarbons might include mono- or polyhalogenated linear, branched or cyclic C1- to C15-alkanes, particularly mono-or polychlorinated or -brominated linear, branched or cyclic C1- to C15-alkanes, preferably mono- or polychlorinated linear, branched or cyclic C1- to C15-alkanes, most preferably methylene chloride or chloroform. Useful examples of aliphatic or cyclic esters might include ethyl acetate, isopropyl acetate, n-butyl acetate or y-butyrolactone. Examples of useful aliphatic or cyclic ketones might include acetone, diethyl ketone or isobutyl methyl ketone or cyclopentanone or isophorone. Examples of cyclic carbonates might include ethylene carbonate, propylene carbonate or mixtures thereof. Examples of aliphatic ethers might include dialkyl ethers, where the alkyl moiety has 1 to 4 carbon atoms. One preferred example is dimethyl ether. Examples of suitable aliphatic alcohols are ethanol, iso-propanol, propanol or butanol.

Suitable oils (i.e. triglycerides) to be used in formulations comprising the bio-based vitamin K2 as defined herein include but are not limited to orange oil, limonene or the like.

Suitable antioxidants to be used in formulations comprising the vitamin K2 as defined herein include but are not limited to water- or fat-soluble antioxidants, e.g. selected from tocopherol, ascorbic acid and/or derivatives thereof as known in the art.

Suitable co-crystallizing agents to be used in formulations comprising the vitamin K2 as defined herein include but are not limited to amino acids, availability enhancers, and small molecules as known in the art.

Suitable coacervates to be used in formulations comprising the vitamin K2 as defined herein include but are not limited to an enzymatic cross-linking step, e.g. achieved via enzymatic bond catalysis.

Methods for the preparation of solid compositions, e.g. solid particles or microcapsules, comprising the bio-based vitamin K2 as defined herein, are known and disclosed in e.g. CN101422446, WO2007045488, or WO2015169816. Particularly useful ingredients for the preparation of such solid vitamin K2 compositions as defined herein are selected from the group consisting of xanthan, gum arabic, gum guar, gelatin, sucrose, mixtures of gelatin/sucrose and/or dextrin, starch, modified food starch, glucose syrup, palm oil, rapeseed oil.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code).

As used herein, the term “riboflavin” also includes riboflavin precursors, flavin mononucleotide (FMN), flavin adenine dinucleotide (FAD), and derivatives thereof. Riboflavin precursors and derivatives of riboflavin, FMN or FAD include but are not limited to: 2,5-diamino-6-ribosylamino-4 (3H)-pyrimidinone-5′-phosphate (DRAPP); 5-amino-6-ribosylamino-2,4 (1H,3H)-pyrimidinedione-5′-phosphate; 2,5-diamino-6-ribitylamino-4 (3H)-pyrimidinone-5′-phosphate; 5-amino-6-ribitylamino-2,4 (1H,3H)-pyrimidinedione-5′-phosphate; 5-amino-6-ribitylamino-2,4 (1H,3H)-pyrimidinedione; 6,7-dimethyl-8-ribityllumazine (DMRL); and flavoproteins. Derivatives of riboflavin include but are not limited to riboflavin-5-phosphate and salts thereof, such as e.g. sodium riboflavin-5-phosphate.

The terms “riboflavin” and “vitamin B2” are used interchangeably herein. The genes involved in biosynthesis of riboflavin as well as methods for fermentative production of riboflavin, in particular fermentative production using Bacillus strains, are known (see e.g. EP405370 or Ullman's Encyclopedia of Industrial Chemistry, 7th Edition, 2007, Chapter Vitamins). These methods may be also applied for production of riboflavin using modified host cells, particularly such as Bacillus, as described herein.

As used herein, the term “vitamin K2” and “menaquinone” (MK-n; wherein “n” is the chain length) is used interchangeably herein. It includes MK-4, MK-5, MK-6, MK-7, MK-9, MK-10, MK-11, MK-12, MK-13 and MK-14, with MK-7 being the most important and preferred isoform.

“Fermentation” or “production” or “fermentation process” as used herein may be the use of growing cells using media, conditions and procedures known to the skilled person, or the use of non-growing so-called resting cells, after they have been cultivated by using media, conditions and procedures known to the skilled person, under appropriate conditions for the conversion of suitable substrates into riboflavin.

The term “bio-based” in connection with the present invention indicates that the products, i.e. fermentation products as defined herein including vitamins, enzymes, oligosaccharides and/or amino acids are produced by biotechnological means and not via chemical synthesis.

The terms “production” or “productivity” are art-recognized and include the concentration of riboflavin formed within a given time and a given fermentation volume (e.g., kg product per hour per liter). The term “efficiency of production” includes the time required for a particular level of production to be achieved (for example, how long it takes for the cell to attain a particular rate of output of a fermentation product). The term “yield” is art-recognized and includes the efficiency of the conversion of the carbon source into the product (i.e., riboflavin). This is generally written as, for example, kg product per kg carbon source. By “increasing the yield and/or production/productivity” of the compound it is meant that the quantity of recovered molecules, or of useful recovered molecules of that compound in a given amount of culture over a given amount of time is increased.

Analytical methods for determining the yield/productivity of riboflavin or vitamin K2 are known in the art. Such methods may include but are not limited to HPLC or use of indicator strains (see e.g. Bretzel et al., J. Ind. Microbiol. Biotechnol. 22, 19-26, 1999).

Particular embodiments of the present invention are as follows:

(1) Process for resource recovery from waste streams in the fermentative production of enzymes, amino acids, oligosaccharides or vitamins, comprising recovery of vitamin K2 from the fermentation waste stream.
(2) Process as of embodiment (1), wherein the recovered vitamin K2 comprises a percentage of at least about 80% (wt/wt) of isoform MK-7.
(3) Process as of embodiment (1) or (2), wherein the percentage of DNA, particularly recombinant DNA, in the fermentation product selected from vitamins, amino acids, oligosaccharides or enzymes is in the range of about 10 ng or less DNA per g of fermentation product, the fermentation product including vitamin K2.
(4) Process as of embodiment (1), (2) or (3), wherein the colony forming unit (CFU) of the production strain, particularly recombinant production strain, per gram of fermentation product is in the range of about 1 CFU or less per 1 g of fermentation product, the fermentation product including vitamin K2.
(5) Process as of embodiment (1), (2), (3), or (4), comprising fermentative production of one or more fermentation products selected from the group consisting of proteases, amylases, glucosidases, cellulases, lysine, tryptophan, monosodium glutamate, riboflavin (vitamin B2), vitamin B5, vitamin B6, vitamin B12, vitamin B3, vitamin B1, vitamin D, and vitamin B7, preferably fermentative production of vitamin B2.
(6) Process as of embodiments (1), (2), (3), (4) or (5) in a host cell selected from the group consisting of Lactococcus, Lactobacillus, Enterococcus, Leuconostoc, Streptococcus, Bacillus, Corynebacterium, Pseudomonas, Flavobacterium, Elizabethingia or Escherichia, preferably selected from B. amyloliquefaciens, B. subtilis or B. licheniformis, B. subtilis natto, B. polymyxa, B. firmus, B. megaterium, B. cereus, B. thuringiensis, Flavobacterium so., Flavobacterium nneningosepticum, Elizabethingia meningoseptica, E. coli, Lactococcus lactis ssp. lactis, Lactococcus lactis ssp. cremonis, Flavobacterium meningosepticum, or Flavobacterium sp. or C. glutannicum.
(7) Process for simultaneous production of two or more fermentation products comprising fermentative production of vitamin K2 being co-produced with one or more primary fermentation products being selected from vitamins, enzymes, oligosaccharides or amino acids, preferably the primary fermentation products being selected from the group consisting of proteases, amylases, glucosidases, cellulases, lysine, tryptophan, monosodium glutamate, riboflavin (vitamin B2), vitamin B5, vitamin B6, vitamin B12, vitamin B3, vitamin B1, vitamin D, and vitamin B7, more preferably selected from vitamin B2.
(8) Process as of embodiments (1), (2), (3), (4), (5), (6) or (7) comprising the steps of:
(a) cultivation of the host cell under suitable conditions and in a suitable culture medium suitable for production of the fermentation product selected from vitamins, oligosaccharides, amino acids or enzymes,
(b) separating the fermentation product from step (a) from the biomass,
(c) recovering of vitamin K2 from the biomass, and optionally
(d) disposal of the waste biomass.
(9) Production of bio-based vitamin K2 comprising the step of recovery/extraction from genetically modified biomass, wherein the recovered vitamin K2 has a content of MK-7 in the range of at least about 80% (wt/wt) and maximal content of about 10 ng DNA, preferably recombinant DNA, per g vitamin K2 as measured by PCR.
(10) Bio-based vitamin K2 product obtained via the process as of embodiment (9) with a content of maximal about 1 CFU per g vitamin K2.
(11) Process as of embodiments (1), (2), (3), (4), (5), (6) or (7), wherein the fermentation product is produced in crystalline form, preferably wherein the fermentation product is vitamin B2, said process comprising the steps of:
(a) fermentation with a suitable host cell, preferably a recombinant host cell, under suitable culture conditions with accumulation of the fermentation product in crystalline form,
(b) separating crystalline fraction from liquid fraction containing the vitamin K2,
(c) extraction of vitamin K2 from the biomass, and optionally
(d) disposal of biomass or further use as fertilizer.
(12) Process as of embodiments (1), (2), (3), (4), (5), (6) or (7), wherein the fermentation product is produced outside the host cell, preferably wherein the fermentation product is enzymes, amino acids, oligosaccharides, said process comprising the steps of:
(a) fermentation with a suitable host cell, preferably a recombinant host cell, under suitable culture conditions with accumulation of the fermentation product outside the host cell,
(b) separating the liquid fraction from the biomass comprising vitamin K2,
(c) extraction of vitamin K2 from the biomass, and optionally
(d) disposal of biomass or further use as fertilizer.
(13) Process as of embodiments (11) or (12), wherein step (c) is performed in the presence of aqueous EtOH-solution with a percentage of at least about 80% (wt/wt) of EtOH.
(14) Process for production of nutritional products comprising the step of mixing bio-based vitamin K2 obtainable by a process as of embodiments (1), (2), (3), (4), (5), (6), (7), (8), (9), (11) or (13) with one or more ingredients selected from the group consisting of encapsulation agents, organic solvent(s), oils, supercritical fluids, antioxidant(s), sugar, co-crystallizing agent(s), coacervate(s), and combinations thereof.

### FIGURES

FIG. 1: Co-extraction of vitamin K2 and riboflavin in B. subtilis. Dotted box indicates vitamin K2 recovery, solid box indicates vitamin B2 recovery.

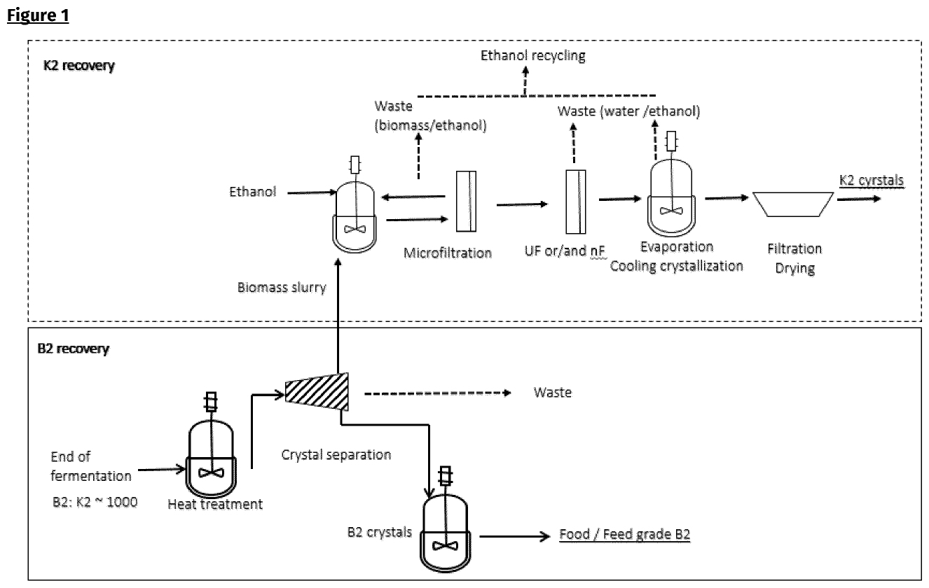

The following examples are illustrative only and are not intended to limit the scope of the invention in any way. The contents of all references, patent applications, patents, and published patent applications, cited throughout this application, particularly EP405370, EP1186664, WO2010052319, WO2004113510, WO2005014594, WO2017036903, WO2007051552, WO20050145949, WO2013124351, EP1814987, CN101422446, WO2007045488, WO2015169816 are hereby incorporated by reference.

### EXAMPLES

### Example 1: General Methods

Unless otherwise mentioned, all media and general methods are disclosed in Sambrook et al. (eds.), Molecular Cloning: A Laboratory Manual. Cold Spring Harbor Laboratory Press: New York (1989) or according to WO2017036903.

Assay of riboflavin production in deep-well microtiter plates (MTP) was performed as follows: an overnight culture was made from a single colony in 3 ml of VY containing selective antibiotics where appropriate. The preculture was incubated at 39° C., 550 rpm, 80% humidity. The next day, 3 ml of RSM were inoculated with the preculture with a starting OD_{600 nm}˜0.05. Cultures in MTP were made in triplicate, covering the wells with a breath seal. MTP were incubated at 39° C., 550 rpm, 80% humidity for 48 hours. 250 μl of the 48 hour-culture were treated with 20 μl of 4M NaOH solution to solubilize the riboflavin crystals (shaken for 1 min at 300 rpm). 230 μl of a 1M potassium phosphate buffer, pH 6.8 were added (shaken for 1 min at 300 rpm). Riboflavin was assayed by HPLC using an Agilent 1100 series HPLC system with a quaternary pump, an autosampler, a UV detector and Fluorescence detector. The separation was achieved using a Supelcosil LC-8 DB (150 mm×4.6 mm×μum). Optimal column temperature was 20° C. The mobile phase was a gradient from 100% 0.1M acetic acid to 50/50 0.1M acetic acid/methanol at 15 minutes for total of 33 minutes per run. The flow rate was 1.0 ml/min and the injection volume set to 5 μl. The UV signal was monitored and used for detection. Calibration range from 0.1 μg/ml to 500 μg/ml. Additionally, the potential accumulation of glucose in the culture broth was analyzed by a Waters HPLC system using a binary pump, an autosampler, a UV- and a refractive index detector. The separation was achieved on a CAPCELL PAK NH2 UG80 column (4.6 mm×250 mm, 5 μm; Shiseido). The optimal column temperature was 40° C. The mobile phase was a mixture of acetonitrile and deionized water at a ratio of 65:35. The flow rate was 1.0 ml/min and the injection volume set to 5 μl or 10 μl. The refractive index signal was monitored and used for detection. The calibration range for each compound was from 0.3 mg/ml to 3 mg/ml.

The analysis of K2 vitamers was performed by HPLC after extraction of the analyte from dried biomass. The biomass was freeze dried and the extraction was carried out by using a Precellys homogenizer. Two disruption cycles were applied. The analysis was carried out on an Agilent 1200 series HPLC system (or similar) using a quaternary pump, an auto sampler and a UV- detector. The separation was achieved on a C18 column, 150×4.6 mm, 3 μm. The optimal column temperature was 15° C. The mobile phase was a mixture of methanol and ethanol, in a 60:40 ratio. Gradient elution was applied ranging from 60:40 MeOH:EtOH to 40:60 in 15 minutes. The flow rate was 1.0 ml/min. The UV signal at 270 nm was monitored and used for quantification.

### Example 2: Fermentative Production of the Primary Fermentation Product in Strains of Bacillus or Corynebacterium and Recovery of Vitamin K2

For riboflavin production as being the primary fermentation product, the protocol according to WO2007051552 has been performed. At the end of the fermentation, the broth was pasteurized and the decanted vitamin B2 crystals (Sedicanter®: Flottweg, Germany) further processed (see e.g. WO20050145949).

For production of pharma-grade riboflavin, crystals were dissolved in concentrated HCL followed by filtration and re-crystallization with water as anti-solvent.

In case of fermentative production of enzymes as primary fermentation product in Bacillus the protocol according to WO2013124351 can be used.

In case of fermentative production of amino acids as primary fermentation product in Corynebacterium the protocol according to EP1814987 can be used.

At the end of the fermentation process, the supernatant (biomass slurry) from the 1^{st} decantation step with Sedicanter® as described above comprising vitamin K2 was treated with ethanol as single extraction solvent. followed by passing through a microfiltration membrane for removal of most of the EtOH and waste biomass. To remove remaining rDNA, proteins or part of lipids still present in the solution, a further purification step was applied, comprising ultrafiltration and/or nanofiltration, with further removal of waste biomass and EtOH. After further evaporation and cooling crystallization, a further filtration and drying step was performed, resulting in purified vitamin K2 crystals with at least 80% (wt/wt) of MK-7 as compared to the less than 1% MK-7 (wt/wt) at the beginning of the treatment as described above. The purity of the menaquinone fraction (containing especially MK-4, MK-5 and MK-6) was also increased from 87 to 88% at the start of the treatment to 97 to 98. Vitamin K2 crystals were analyzed for their rDNA content, which was measured below 10 ng/g product by PCR.

The ratio of riboflavin to vitamin K2 (MK-7) obtained with this procedure as described above was in the range of 1000 to 1.

### Example 3: Solid Vitamin K2 Formulation

Vitamin K2 crystals with at least 80% MK-7 (wt/wt) is dissolved in medium chain triglyceride oil at 62° C. Gum acacia and sugar are dissolved in water and the oil-phase comprising vitamin K2 is added at 62° C.

After homogenization (e.g. in a rotor/stator or high pressure homogenization) the resulting dispersion can be spray-dried including a thin starch coating. Ideally, the final size of the droplets is 100-800 nm.

Thus generated vitamin K2 microcapsules can further be mixed with dietary minerals (e.g. MgO, CaCO₃) microcrystalline cellulose powder, Mg-stearate or the like in order to produce solid tablets, as exemplified in WO2015169816 (Ex. 1 to 7).

1. Process for co-fermentation of at least two fermentation products in a suitable host cell capable of co-production of a primary fermentation product and a secondary fermentation product, wherein one fermentation product is water-soluble and one fermentation product is fat-soluble organic compound, and wherein the fat-soluble fermentation product is preferably a fat-soluble vitamin, more preferably vitamin K, most preferably vitamin K2.

2. Process according to claim 1, wherein the primary fermentation product is water-soluble and the secondary fermentation product is fat-soluble.

3. Process according to claim 1, wherein the primary and the secondary fermentation products are selected from vitamins.

4. Process according to claim 1, wherein the primary fermentation product is selected from the group consisting of vitamin B2, vitamin B5, vitamin B6, vitamin B12, vitamin B3, vitamin B1, vitamin B7, preferably vitamin B2, B5, B6, B12, more preferably vitamin B2.

5. Process according to claim 1, wherein the secondary fermentation product is extracted from the biomass generated during the fermentative production of the primary fermentation product.

6. Process according to claim 1, wherein the secondary fermentation product is vitamin K2, preferably vitamin K2 with a percentage of at least about 80% (wt/wt) of MK-7.

7. Process according to claim 1, wherein the secondary fermentation product is vitamin K2 with a percentage of about 10 ng or less DNA per g of fermentation product, preferably per g of vitamin K2.

8. The process according to claim 1, wherein the secondary fermentation product is vitamin K2 with a percentage of less than about 1 colony forming unit (CFU) production strain per 1 g of fermentation product, preferably per g of vitamin K2.

9. The process according to claim 1, in a host cell selected from the group consisting of Lactococcus, Lactobacillus, Enterococcus, Leuconostoc, Streptococcus, Bacillus, Corynebacterium, Pseudomonas, Flavobacterium, Elizabethingia and Escherichia, preferably selected from B. amyloliquefaciens, B. subtilis, B. licheniformis, B. subtilis natto, B. polymyxa, B. firmus, B. megaterium, B. cereus, B. thuringiensis, Flavobacterium sp., more preferably Flavobacterium meningosepticum, Elizabethingia meningoseptica, E. coli, Lactococcus lactis ssp. lactis, Lactococcus lactis ssp. cremonis, Flavobacterium meningosepticum or C. glutamicum, most preferably from Bacillus subtilis.

10. Process according to claim 1 comprising the steps of:
(a) cultivation of the host cell under suitable conditions and in a suitable culture medium suitable for production of the primary fermentation product, preferably water-soluble vitamin,
(b) isolation of the primary fermentation product from step (a) from the production stream,
(c) recovery of the secondary fermentation product, preferably vitamin K2 from the biomass generated during step (a), and optionally
(d) purification of the primary and/or secondary fermentation product.

11. Process for production of bio-based vitamin K2 according to claim 1 comprising the step of recovery/extraction from genetically modified biomass, wherein the recovered vitamin K2 has a content of MK-7 in the range of at least about 80% (wt/wt) and maximal content of about 10 ng DNA, preferably recombinant DNA, per g vitamin K2 as measured by PCR.

12. Process according to claim 1 comprising extraction of vitamin K2 as secondary fermentation product in a hexane-free solvent, preferably in an aqueous solution with at least about 80% (wt/wt) ethanol.

13. Process according to claim 1 further comprising production of a nutritional product comprising mixing the bio-based fermentation product(s) with one or more ingredients selected from the group consisting of encapsulation agents, organic solvent(s), oils, supercritical fluids, antioxidant(s), sugar, co-crystallizing agent(s), coacervate(s), and combinations thereof.

14. Bio-based vitamin K2 with a content of about 1 CFU or less production strain per g vitamin K2 obtainable by a process according to claim 1.
